(19) United States
(12) Patent Application Publication
(10) Pub. No.: US 2024/0238755 A1
(43) Pub. Date: Jul. 18, 2024

(54) Products for treating inflammations, infections, diseases, and malodors. containing adsorbent porous carbonaceous material

(71) Applicant: Angela Jean Yonce, Petal, MS (US)

(72) Inventor: Angela Jean Yonce, Petal, MS (US)

(73) Assignee: Angela Jean Yonce, Petal, MS (US)

(21) Appl. No.: 18/607,507

(22) Filed: Mar. 17, 2024

Related U.S. Application Data
(63) Continuation of application No. 16/932,758, filed on Jul. 18, 2020.

(51) Int. Cl. B01J 20/20; A61F 13/15; A61L 15/08; A61L 15/14; A61L 15/18; A61L 15/46; A61L 26/00; B01J 20/28; A61F 13/20; A61L 15/20; A61L 15/34

(52) U.S. Cl. CPC B01J 20/20; A61F 13/15; A61L 15/08; A61L 15/14; A61L 15/18; A61L 15/46; A61L 26/0004; B01J 20/28028; B01J 20/2805; A61F 13/2074; A61L 15/20; A61L 15/34; A61L 2209/22; A61L 2300/108; A61L 2300/404

(57) ABSTRACT

The present invention includes the composition and manufacturing of a product to treat various types of infections, and diseases ailments. The invention embodies a disposable absorbent or nonabsorbent article comprising an unbound/freely associating adsorbent porous carbonaceous material for the entrapment and/or treatment of pathogens and adverse biological by-products. The product form includes three dimensional carriers such as tampons, panty liners, incontinence pads, diapers, sanitary towels, swabs, cotton balls, pouches, and/or plugs, which contain adsorbent porous carbonaceous material that effectively reduces and/or eliminates adverse situations sourcing from pathogens and harmful biological by-products, including fungi, bacteria, viruses, and parasites. The article is used to treat yeast infections, vaginitis, sexually transmitted diseases, tooth infections, abscess, nail-bed and cuticle infections, and other ailments by way of internal insertion and/or completely covering of the body cavity, mucous membrane, and/or affected areas, creating a clear and direct path for contact with pathogens and adverse biological by-products.

### BACKGROUND

The disruptive innovation is a cost-effective, over the counter, pharmaceutical-free product that can improve health by reducing various types of inflammations, infections, and diseases by enabling a low-risk self-treatment with better effectiveness and less adverse side effects. Specifically, it includes a disposable absorbent or nonabsorbent article containing unbound/freely associating adsorbent porous carbonaceous material for the entrapment and/or treatment of odor, pathogens, and adverse biological by-products.

In the case of women's health, diseases such as these can have detrimental effects on reproductive health and unborn children. It is well documented that for a variety of reasons, these conditions go largely undetected or untreated. The invention, when incorporated with women's menstrual products, avoids undesirable treatment alternatives such as creams and antibiotics and can act as a preventative for women's health as a normal part of menstruation products.

As related to field-deployed soldiers, minimizing infection in bullet wounds with improved self-treatment options until proper medical attention can be administered is of paramount importance to improve chances of recovery.

### SUMMARY OF INVENTION

The disruptive innovation is a cost-effective, over the counter, pharmaceutical-free product that can improve health by reducing various types of inflammations, infections, and diseases by enabling a low-risk self-treatment with better effectiveness and less adverse side effects. Specifically, it includes a disposable absorbent or nonabsorbent article containing unbound/freely associating adsorbent porous carbonaceous material for the entrapment and/or treatment of odor, pathogens, and adverse biological by-products.

Treatment Method for the Removal of Disease-Causing Pathogens and/or Harmful Biological-by Products for Medical and/or Dental Related Conditions.

### DETAILED DESCRIPTION

The invention offers an over the counter (OTC), easily deployable, all-natural prophylactic and/or treatment of odor and infectious diseases. It is a low-risk self-treatment with better effectiveness, less adverse side effects, without doctor intervention or prescription. It is a cost-effective over the counter, pharmaceutical-free product that improves health by reducing various types of inflammations, infections, and diseases. The invention works by way of contact with body orifices, flesh wounds, and other effected areas, by adsorption of fungi, bacteria, viruses, adverse biological by-products, parasites, and include sexually transmitted diseases (STDs).

The product may be effectively used for conditions including but not limited to malodor, bacterial vaginosis, vaginitis, urinary tract infections, viral infections, yeast infections, sexually transmitted diseases, nail fungal infections, tooth infections, bullet wounds, scrapes, blisters, and abrasions.

The uniqueness of the product is embodied in the improved effectiveness and the wide array of pathogens and harmful biological side products which it can render inactive. The improved effectiveness is due to the unbound nature of the carbonaceous material, for maximum contact with harmful compounds, and the wide array of pathogens susceptible is due to the selective and adsorptive nature of the carbonaceous material. The lack of negative side effects can be traced back to the minimal and innocuous formulation ingredients, which selectively adsorb or trap harmful compounds over healthful compounds, and do not have ‘extra’ ingredients that can harm health-supporting compounds or create other adverse chemical reactions and effects.

Loosely coating, saturating or filling any textile or nonwoven material, inside and/or outside with adsorbent porous carbonaceous material creates sufficient presence for effectiveness. The less compressed, the better the effectiveness. This may be achieved via mixing, spraying, dip-coating, powder-coating, solution-coating, dredging, submersing, tumbling, shaking, compounding, and extrusion techniques at any stage of the production process. The latter of which meaning at initial creation of raw materials, such as fiber or thread spinning, to post-processing of commercial product forms.

While many claim adsorbent effectiveness is due to the specific pore size of activated carbon, which is related to source and processing conditions, we contend that the broad spectrum effectiveness of our product is due to a wide distribution of pore sizes, and that multiple mechanisms enable such broad effectiveness. One additional mechanism includes but is not limited to the physical entrapment of flagellum-like species. Likewise, other allotropes of carbon (similar carbonaceous materials in the same family), whether amorphous or not, have the capability to behave in similar (multiple) ways. These materials include activated charcoal/carbon, carbon black, un-activated charcoal/carbon, graphene, and graphite in powdered and granular forms and mixtures thereof. In the case of activated carbon, activated carbon can be derived from, but not limited to, bamboo, coconut husk, willow peat, hardwood, softwood, coir, lignite, coal, and petroleum pitch. ‘Activated’ carbon becomes ‘activated’ by heating at elevated temperature and pressure in the presence of steam, other gases such as argon or nitrogen, or strong acids and bases, or hydroxides, or chlorides. It is generally accepted that processing conditions and carbon source are key variables that impart the resulting pore size. For this work, sources of activated carbon include but are not limited to industrial grade activated carbon from hardwoods and coal, and medical grade ingestible activated carbon from coconut shells.

The invention includes an absorbent or nonabsorbent material in any form, that contains adsorbent porous carbonaceous material for the entrapment and/or treatment of odor, pathogens, and adverse biological by-products that include but are not limited to fungi, bacteria, viruses, parasites, and harmful biological by-products, including sexually transmitted diseases (STD), by way of adsorption via contact with body orifices, flesh wounds, and other affected areas.

The product forms include, but are not limited to tampons, panty liners, incontinence pads, diapers, sanitary towels, swabs, balls, pouches, paste-encased casts, bandages and/or plugs.

The product is used by inserting internally and/or completely covering the body orifice, wound, nailbeds, cuticles, or otherwise affected areas creating a clear and direct path by which the pathogens, adverse biological by-products, and odor-causing compounds will come in contact with the adsorbent porous carbonaceous material for entrapment and/or treatment. By way of the product contact with body orifices, flesh wounds, and/or other affected areas, the pathogens are rendered ineffective.

The invention has presented no adverse effects due to the adsorptive nature of the porous carbonaceous material and also because it does not inhibit the action of ‘good’ bacteria, which allow normal healing mechanisms to resume. While the adsorbent porous carbonaceous material renders pathogens inactive and ineffective, it also balances pH.

In certain persistent fungal infections, such as those that infect fingernails and toenails, it has been discovered that combining a paste of carbonaceous material with chosen essential oils, one of which includes but is not limited to, pine oil, and treating the affected area by sealing with a bandage has provided dramatic improvement and restoration of healthy nails.

To date, the invention has been used to adsorb and treat odor and infections, stemming from fungi, yeast, mold, bacteria, viruses, and/or parasitic pathogens, including biological proxies/surrogates of sexually transmitted diseases. In vitro and in vivo experiments have been conducted and support effectiveness. While in vitro experiments are still underway, select volunteer in vivo experiences are recounted below:

Case study 1: Volunteer (person's identity disclosed to the inventor) had a urinary tract infection (UTI) and was experiencing a burning sensation during urination. The subject began to use the treatment tampon on the second day of symptoms, for three successive 4-hour durations, and continued treatment on the third day with two successive 4-hour durations. Immediately after the first application the burning sensation began to diminish. The subject's symptoms completely vanished after the third application and subject has not had any other symptoms since the treatment.

Case Study 2: Volunteer's symptoms began in November 2019 as a sharp pain when urinating with some redness in the area and general discomfort and well as itchiness. The symptoms occurred in phases that would last from 1 day to multiple weeks. Approximately 6 months later the subject began having stomach issues, beginning with lack of appetite, changes in bowel movement frequency, and regular diarrhea. The stomach issues as well as the pain in the vaginal area continued for 2 months and lead to a 201b weight loss over that time period. The Volunteer began using the treatment tampon Jun. 30, 2020 and used it daily for 9 days. The pain during urination was completely resolved and stomachache and bowel movements after the first usage. After use on the 2nd day, her appetite began to return and continued to increase. By the 9th day of use of the treatment tampon, her appetite completely returned, and the volunteer began to gain weight. Subject left the tampon in around 7 to 10 hours per usage. Only one slight vaginal pain occurred on day 5, but it was resolved by day 6.

Case Study 3: An avid bicyclist and runner complained of vaginal malodor after long periods of cycling and/or running. Prior to the next training activity, she used a treatment tampon. The volunteer reported that all odor was eliminated by using the treatment tampon during the 2-hour intense exercise period, which had never occurred before.

Case Study 3: A volunteer realized she was getting a yeast infection but could not get to a doctor. After 4 hours of using a treatment tampon, the symptoms were eliminated and did not return.

Case Study 4: After being told that surgery would be required, along with a costly antibiotic, for a severe tooth infection, the inventor created a pouch containing the carbonaceous material, and tucked it in contact with the swollen gum and infected tooth. After 8 hrs. of treatment, within 24 hrs the swelling and infection completely resolved, and surgery was no longer required.

Case Study 5: After multiple rounds of medical treatment for fungal infection on toenails, with no improvement, a volunteer used a paste mixture of carbonaceous material with essential oil and treated overnight the affected areas, sealing with a bandage. After 1 round of overnight treatment, the nailbeds were noticeably improved and over the course of 6 months, healthy toenails replaced the fungal-deformed toenails.

Case Study 6: A volunteer that was particularly susceptible to vaginal bacterial infections used a treatment tampon at the onset of symptoms. It was administered for 8 hours and volunteer reported complete elimination of symptoms.

I claim:

1. A treatment method for the removal of disease-causing pathogens and/or harmful biological by-products for medical and/or dental related conditions, the treatment method including adsorption, entrapment, and extraction of disease-causing pathogens and/or harmful biological by-products from a living being, the method comprising:
providing the treatment method for medical and/or dental related conditions comprising: (a) an unbound, freely associating adsorbent porous carbonaceous material: (b) a woven and/or nonwoven, and/or absorbent and/or nonabsorbent textile material;
insertion and/or a complete covering of an orifice, mucus membrane, fingernail, toenail and/or an infected area with the textile material containing the unbound, freely associating adsorbent porous carbonaceous material;
preventing external matter from entering the orifice, mucus membrane, fingernail, toenail and/or infected area post insertion and/or a complete covering of the orifice, mucus membrane, fingernail, toenail and/or infected area by sealing the textile material containing the unbound, freely associating adsorbent porous carbonaceous material with a bandage;
treating the medical and/or dental related conditions using the textile material containing the unbound, freely associating, adsorbent porous carbonaceous material for an administration duration sufficient for the unbound, freely associating, adsorbent porous carbonaceous material to adsorb, entrap, and extract the disease-causing pathogens from the living body; and
removing the inserted and/or complete covering of the textile material, from the orifice, mucus membrane, fingernail, toenail and/or the infected area, wherein the unbound, freely associating, adsorbent porous carbonaceous material includes adsorbed, entrapped, and extracted the disease-causing pathogens and/or harmful biological by-products from the living body when the inserted and/or complete covering textile material has been removed.

2. The treatment method of claim 1, wherein the unbound/freely associating adsorbent porous carbonaceous material: (a) comprises at least one of: activated charcoal/carbon: carbon black, an un-activated charcoal/carbon; a graphene; other allotropes of carbon; whether amorphous or not (b) is provided in a granular or powder form; and wherein the treatment method further comprises at least one of bamboo; coconut husk; hardwood; softwood; willow peat; lignite and petroleum pitch (c) is heated at elevated temperature in the presence of a steam, gas argon and/or nitrogen; strong acid; base; hydroxide; and/or a chloride

3. The treatment method of claim 1, wherein the treatment method further comprises: a natural and/or a synthetic material; cotton, wool, polyurethane; nylon; and/or polyester.

4. The treatment method of claim 1, wherein the providing step comprises one or more of injecting; mixing; spraying; dip-coating; powder-coating; solution-coating; dredging; submersing, tumbling; shaking; compounding; infusion; and/or extrusion of the unbound, freely associating, adsorbent porous carbonaceous material to the textile material.

5. The treatment method of claim 1, wherein the disease-causing pathogens and/or harmful biological by-products are selected from bacteria; yeast; virus, parasite; or a fungus; and wherein the treatment method of medical and/or dental related condition is selected from; a urinary tract infection; a yeast infection; a Chlamydia infection; a vaginosis; a trichomoniasis infection; a parasite infection; a fungal infection, a sexually transmitted disease, an abscess, a tooth infections, gum infection, mouth sores, mouth ulcers, any wounds, abrasions, blisters and/or scrapes.

6. The treatment method of claim 1, wherein the administration duration is about: 4-10 hours duration for yeast infections, and/or 8-10 hours for non-yeast infections, of the medical and/or dental related conditions.

7. The treatment method of claim 1, wherein the textile material containing unbound, freely associating, adsorbent porous carbonaceous material is capable of being inserted onto a body interface for the adsorption, entrapment, and extraction of the disease-causing pathogens and/or harmful biological by-products from the living being.

8. The treatment method of claim 1, Wherein the textile material containing unbound, freely associating, adsorbent porous carbonaceous material further comprises at least one essential oil.

9. The treatment method of claim 1, wherein treating the medical and/or dental related condition further comprises eliminating an unpleasant smell or malodor through the adsorption, entrapment, and extraction of the disease-causing pathogens and/or harmful biological by-products from the living body.

10. The treatment method of claim 1, wherein the bandage further comprises a feminine sanitary napkin; gauze, skin wraps, and/or a band aid.

11. The treatment method of claim 10, wherein the feminine sanitary napkin; gauze, skin wraps, and/or a band aid further comprises the additional unbound, freely associating, adsorbent porous carbonaceous material.
